(19) United States
(12) Patent Application Publication
(10) Pub. No.: US 2025/0135174 A1
(43) Pub. Date: May 1, 2025

(54) Port Identification Insert and Assembly Alignment Feature

(71) Applicant: Bard Peripheral Vascular, Inc., Franklin Lakes, NJ (US)

(72) Inventors: Bryon Ray Densley, Rochester, MN (US); Christian Andersen, Queen Creek, AZ (US); Jason R. Stats, Layton, UT (US)

(21) Appl. No.: 18/730,587

(22) PCT Filed: Jan. 21, 2022
(86) PCT No.: PCT/US22/13368

§ 371 Date: Jul. 19, 2024

(51) Int. Cl. A61M 39/02

(52) U.S. Cl. CPC A61M 39/0208; A61M 2039/0238; A61M 2205/32; A61M 2207/00

(57) ABSTRACT

Embodiments disclosed herein are directed to an identification insert and assembly alignment feature for a subcutaneous access port. The port can include an identification insert assembled with the device and configured to be visible under medical imaging when placed subcutaneously. The insert can indicate a property of the port and a location or orientation of the port to facilitate access thereto. An alignment feature can be configured to align the insert with the port in a first orientation during manufacture. The alignment feature can also be configured to misalign the insert with the port in a second orientation, different from the first orientation. This can prevent the port from being assembled if the insert is coupled with the port incorrectly. A port having an insert assembled incorrectly can lead to unnecessary disruptions and complications to the patient to correct the problem.

### SUMMARY

Briefly summarized, embodiments disclosed herein are directed to an identification insert, or marker, and an assembly alignment feature for a subcutaneous access port. A subcutaneous vascular access device, or “port,” can include an identification marker assembled with the device and designed to be visible under one or more medical imaging techniques (e.g. x-ray, fluoroscopy, ultrasound, MRI, CT, etc.) when the port is placed subcutaneously. The marker can indicate to a user a property of the port and can indicate a location and orientation of the port to facilitate access thereto. Various identification inserts have been developed that align with a septum of the device to indicate a target location. Other port designs include identification inserts positioned in a nose portion, disposed opposite a stem portion, so as not to be obscured when accessing the septum. However, some of these identification markers can be assembled with the port incorrectly. As such, when imaging the subcutaneous port under medical imaging, the identification marker appears to indicate that the port has flipped or is otherwise incorrectly positioned when the port is instead correctly positioned. This can lead to unnecessary disruptions and complications to the patient to correct the problem. Disclosed herein are devices and methods that address the foregoing.

The following items also define the invention. Disclosed herein is a subcutaneous access port including, a base defining a reservoir, a stem in fluid communication with the reservoir and extending longitudinally (i.e. in a longitudinal direction), a septum positioned at least partially over the reservoir, a cap configured to engage the base and secure the septum over the reservoir, the cap including a nose portion (optionally opposite the stem and) defining a recess, an alignment feature including a protrusion extending from a surface/perimeter/edge of the recess into the recess (and for aligning the cap and a radiopaque insert), and a radiopaque insert including an aperture, a perimeter of the aperture defining a symbol, the radiopaque insert configured to fit within the recess in a first orientation, the (access port, in particular the radiopaque insert, configured such that the) protrusion maintains the radiopaque insert in the first orientation and prevents the radiopaque insert from engaging/being received in the recess in a second orientation different from the first orientation.

In some embodiments, the base engages the cap to secure the radiopaque insert within the recess in the first orientation.

In some embodiments, one of the protrusion or the base abuts against the radiopaque insert in the second orientation and prevents the cap from engaging the base.

In some embodiments, the protrusion engages a notch disposed in a perimeter of the radiopaque insert in the first orientation, and the access port is configured such that the notch abuts against a portion of the perimeter of the radiopaque insert in the second orientation.

In some embodiments, the protrusion is configured to engage the aperture of the radiopaque insert in the first orientation and abut against a portion of the radiopaque insert in the second orientation.

In some embodiments, the symbol is configured to indicate a property of the port.

In some embodiments, the symbol includes the letters “CT”.

In some embodiments, the insert is disposed on an opposite side of the reservoir from the stem, along a longitudinal axis.

In some embodiments, the reservoir is disposed between the stem and the insert along a longitudinal axis.

In some embodiments, the nose portion extends from the port along the longitudinal axis, opposite the stem.

Also disclosed is a method of manufacturing a subcutaneous access port including, providing a cap including a nose portion defining a recess, placing a radiopaque insert within the recess in a first orientation, the radiopaque insert including an aperture, a perimeter of the aperture defining a symbol, engaging an alignment feature with the radiopaque insert, the alignment feature extending/protruding from a surface of the cap into the recess to maintain the radiopaque insert in the first orientation and preventing the radiopaque insert from engaging the recess in a second orientation different from the first orientation, and coupling a base with the cap, a portion of the base extending across the recess to secure the radiopaque insert within the recess in the first orientation.

In some embodiments, the base defines a reservoir and includes a stem extending therefrom along a longitudinal axis, the stem in fluid communication with the reservoir.

In some embodiments, the method further includes securing a septum over the reservoir when/while the base is coupled with the cap.

In some embodiments, one of the alignment feature or the base is configured to abut against the radiopaque insert in the second orientation and to prevent the cap from engaging the base.

In some embodiments, the alignment feature engages a notch disposed in a perimeter of the radiopaque insert, the alignment feature engages the notch in the first orientation, and abuts against a portion of the perimeter of the radiopaque insert in the second orientation.

In some embodiments, the alignment feature is configured to engage the aperture of the radiopaque insert in the first orientation and abut against a portion of the radiopaque insert in the second orientation.

In some embodiments, the symbol indicates a property of the port.

In some embodiments, the symbol includes the letters “CT”.

In some embodiments, the insert is disposed on an opposite side of the reservoir from the stem, along a longitudinal axis.

In some embodiments, the reservoir is disposed between the stem and the insert along a longitudinal axis.

In some embodiments, the nose portion extends from the port along the longitudinal axis, opposite the stem.

In some embodiments a method is disclosed for manufacturing access devices according to any of the aforementioned items.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective top-side view of a port including an identification marker, in accordance with embodiments disclosed herein.

FIG. 1B shows a perspective under-side view of a port including an identification marker, in accordance with embodiments disclosed herein.

FIG. 2 shows an exploded under-side view of a port including an identification marker, in accordance with embodiments disclosed herein.

FIG. 3 shows an exploded under-side view of a port including an identification marker, in accordance with embodiments disclosed herein.

FIG. 4A shows a perspective under-side view of a base of a port, in accordance with embodiments disclosed herein.

FIG. 4B shows a perspective under-side view of a cap of a port including an identification marker, in accordance with embodiments disclosed herein.

FIG. 5A shows close up detail of a recess including an alignment feature, in accordance with embodiments disclosed herein.

FIG. 5B shows close up detail of a recess including an alignment feature engaged with an identification marker, in accordance with embodiments disclosed herein.

FIG. 6 shows a lateral cross-section view of a nose portion of a port including an identification marker, in accordance with embodiments disclosed herein.

FIGS. 7A-7B show exemplary identification markers, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

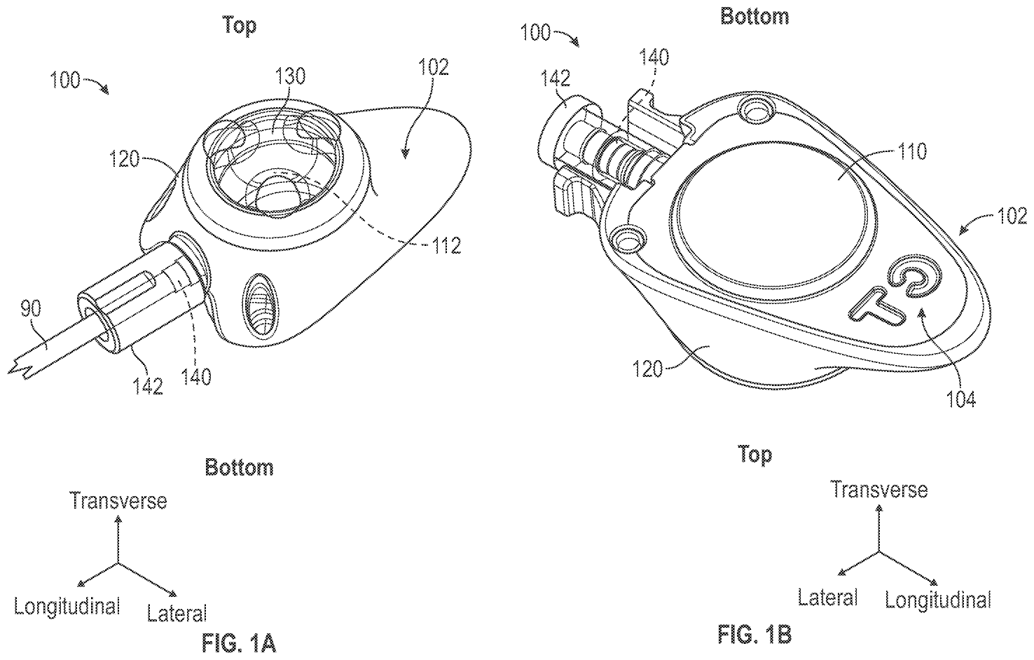

To assist in the description of embodiments described herein, as shown in FIGS. 1A-1B, a longitudinal axis extends substantially parallel to an axial length of the port stem. A lateral axis extends normal to the longitudinal axis, and a transverse axis extends normal to both the longitudinal and lateral axes. A horizontal plane is defined by the longitudinal and lateral axes. A vertical plane extends perpendicular to the horizontal axis.

Unless defined otherwise, all technical and scientific terms used herein have the same meaning as commonly understood by those of ordinary skill in the art.

FIGS. 1A-1B show an exemplary subcutaneous access device, or “port” 100. FIG. 1A shows a top-side perspective view of the port 100. FIG. 1B shows an under-side perspective view of the port 100. The port 100 can generally include a base 110 that defines a reservoir 112, and includes a stem 140 extending therefrom along a longitudinal axis. The stem 140 can define a stem lumen that is in fluid communication with the reservoir 112. A needle penetrable septum 130 can be disposed over the reservoir 112 and can be secured in place by a cap 120. The septum 130 can be formed of a silicone rubber or similar suitable material. In use, when the port 100 is placed subcutaneously, a top side of the port 100 would be disposed proximate a skin surface. A user can penetrate the skin using an access needle (not shown) and can penetrate a septum 130 to access the reservoir 112.

The cap 120 can engage the base 110 in an ultrasonically welded, press-fit, interference fit, or snap fit engagement. In an embodiment, the cap 120 can be secured to the base 110 using adhesive, bonding, welding, ultrasonically welded, or similar suitable means. In an embodiment, the cap 120 can be over-molded on to the base 110. In an embodiment, the cap 120 and the base 110 can be formed as a single monolithic piece formed of the same material.

In an embodiment, the base 110 can be formed of a substantially rigid or resilient material including, but not limited to, a plastic, polymer, thermoplastic, metal, alloy, composite, combinations thereof, or the like. In an embodiment, the cap 120 can be formed of a softer material relative to the base 110 including, but not limited to, a plastic, polymer, thermoplastic, elastomer, rubber, silicone rubber, metal, alloy, composite, combinations thereof, or the like. In an embodiment, the base 110 and the cap 120 can be formed of the same material and can display the same mechanical properties. In an embodiment, a catheter 90 can engage the stem 140 in one of a press-fit or interference fit engagement. Optionally a cathlock 142 can engage one or both of the catheter 90 and the stem 140 to further secure the catheter 90 to the stem 140.

In an embodiment, the port 100 can include a nose portion 102 extending from the port 100 along a longitudinal axis and disposed opposite the stem 140 across a central transverse axis of the reservoir 112. The nose portion 102 can define a substantially wedge shaped profile extending from the port 100 configured to facilitate subcutaneous placement thereof. For example, as the port 100 is urged subcutaneously, the nose portion 102 can facilitate separation of subcutaneous tissues to form a tissue pocket.

In an embodiment, the nose portion 102 can include a symbol 104, e.g. an alphanumeric symbol, shape, icon, or the like, configured to indicate a property of the port 100. For example, as shown in FIG. 1B, a bottom surface of the base 110 can include a symbol 104 such as the letters “CT” which is designed to indicate a property of the port 100 such as the port 100 is configured for high pressure infusion, or the like. These and other properties of the port 100 including make, model, part number, serial number, website addresses, bar codes, QR codes, combinations thereof, or the like are contemplated to fall within the scope of the present invention. The symbol 104 can be printed, embossed, or engraved onto or into the base 110 and can be directly observable by a user to visually indicate one or more properties of the port 100. As will be appreciated the port 100 can include one or more symbols 104 disposed on one or more surfaces of the port 100, without limitation.

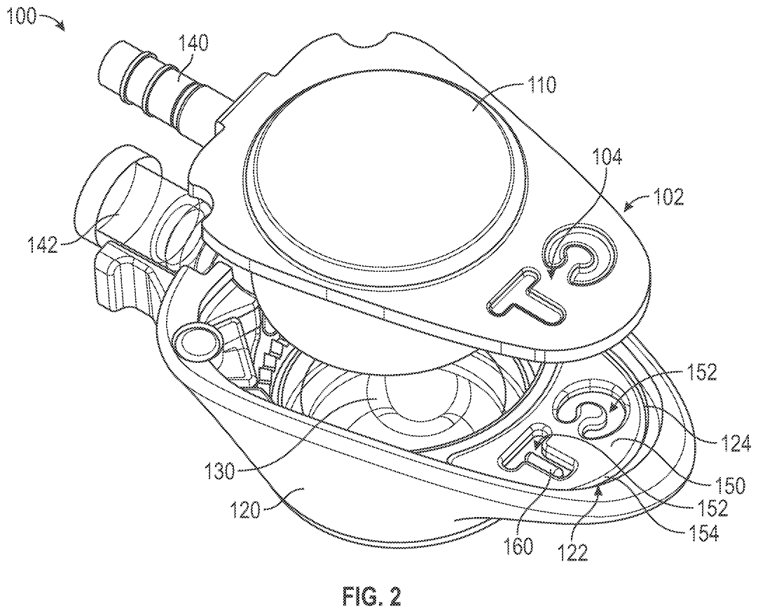

FIG. 2 shows an exploded view of the port 100 of FIG. 1B with the base 110 separated from the cap 120. In an embodiment, one or both of the base 110 and the cap 120 can include a recess 122 disposed within the nose portion 102 of the port 100 and extending over a horizontal plane. The recess 122 can also extend transversely to define a depth (d). In an embodiment, a perimeter 124 of the recess 122 can define curvilinear or polygonal shape extending over the horizontal plane. As shown, the perimeter 124 can define a substantially crescent shape 124, or “lune.” However, it will be appreciated that other curvilinear or polygonal shapes are also contemplated.

In an embodiment, the port 100 can further include an identification marker, or identification insert (“insert”) 150 configured to fit within the recess 122. The insert 122 can be formed of a radiopaque material configured to be visible when imaged by one or more medical imaging techniques. Exemplary medical imaging techniques can include, but not limited to, x-ray, fluoroscopy, ultrasound, magnetic resonance imaging (MRI), computed tomography (CT), positron emission tomography (PET), combinations thereof, or the like.

In an embodiment, a shape of an outer perimeter 154 of the insert 150 can mirror that of the outer perimeter 124 of the recess 122. For example, as shown in FIG. 2, the outer perimeter of the insert 154 can define a crescent shape, or “lune,” equal to or slightly smaller than the crescent shape of the outer perimeter 124 of the recess 122. It will be appreciated, however, that other curvilinear or polygonal shapes are also contemplated. In an embodiment, the insert 150 can be secured within the recess 122 using one of adhesive, bonding, welding, or the like. In an embodiment, the outer perimeter of the insert 154 can define a shape equal to or slightly larger than the shape of the outer perimeter 124 of the recess 122. As such, the insert 150 can engage the recess in a press-fit, interference fit, or snap-fit engagement.

In an embodiment, the insert 150 can further include one or more can include one or more apertures 152 extending through the insert 150 along a transverse axis. A perimeter of the aperture 152 can define a symbol such as an alphanumeric symbol, shape, icon, or the like, as described herein. As such, the aperture 152 can define a “punch-out” symbol 152, and can be termed a “negative” insert. By contrast, a “positive” insert can include an outer perimeter that defines one or more symbols.

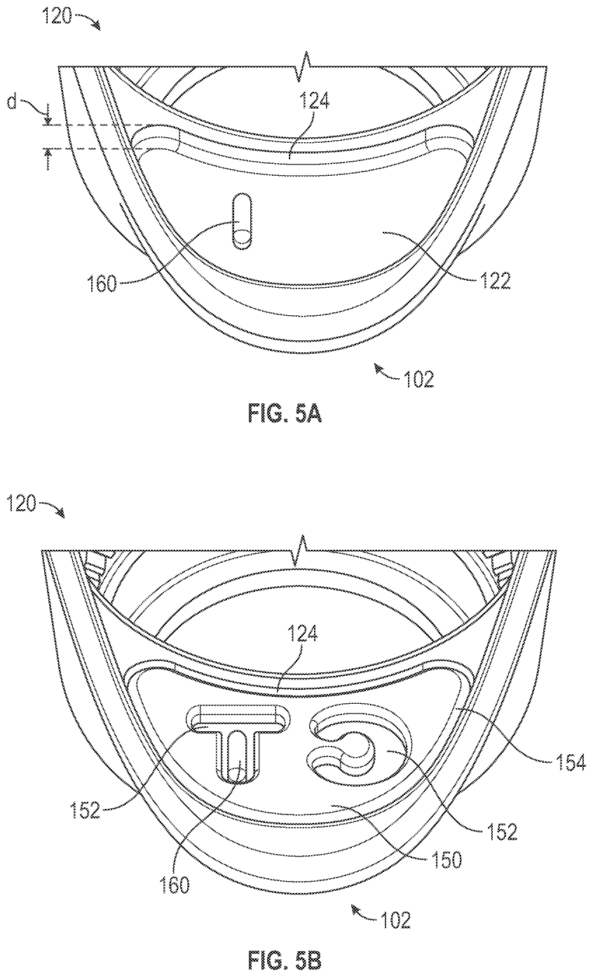

As shown in FIGS. 2-3, 4B, 5B and 7A-7B, the aperture 152 can define one or more letters, such as “CT.” As such, when the negative insert 150 is imaged using medical imaging techniques, portions of the insert 150 that are radiopaque can contrast with the perimeter of the apertures 152 and the perimeter 154 of the insert 150 to define the symbol. The insert 150 can then indicate one or more properties of the port 100, a location, or an orientation of the port 100 when imaged subcutaneously.

In an embodiment, the port 100 can further include an alignment feature 160. The alignment feature 160 can be configured to engage a portion of the insert 150 and can align the insert 150 with the recess 122 in a first orientation in three dimensional space. Further, the alignment feature 160 can also be configured to prevent the insert 150 from engaging the recess 122 in a second orientation, different from the first orientation, in three dimensional space.

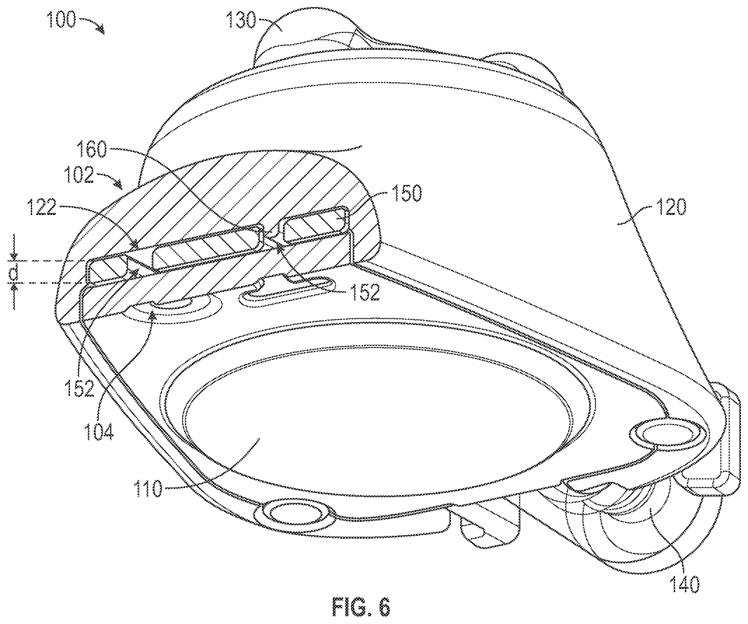

In an embodiment, as shown in FIGS. 2, 4B, and 5A-6, the alignment feature 160 can include a protrusion extending transversely into the recess 122 from one of the cap 120 or the base 110 by a distance of equal to, or less than, a depth (d) of the recess 122. The alignment feature 160 can be positioned within the recess 122 so as to engage an aperture 152. For example, as shown in FIG. 2, the alignment feature 160 includes a linear protrusion extending into the recess 122 and extending along a longitudinal axis. The alignment feature 160 can be configured to engage a “T” shaped aperture 152. However, it will be appreciated that the alignment feature 160 can extend linearly or curvilinearly, longitudinally, laterally, and/or at an angle there between and can engage one or more portions of the aperture(s) 152. For example, the alignment feature can engage one or more of the cross portion of the “T” shaped aperture 152 or a curved portion of the “C” shaped aperture 152.

In an embodiment, the port 100 can include two or more alignment features 160 configured to engage one or more apertures 152. For example, the alignment feature(s) 160 can include one or more posts extending transversely into the recess 122 and defining a circular or polygonal horizontal cross-sectional shape. The one or more alignment features 160 can be positioned within recess 122 to engage a first aperture 152. These and other numbers and combinations of alignment features 160 are contemplated to fall within the scope of the present invention.

In an embodiment, a portion of the base 110 can extend over at least a portion of the recess 122 and can secure insert 122 within the recess in the first orientation. As shown in FIG. 2, a portion of the base 110 can extend over the entire recess 122 to seal the insert 150 within the recess 122. The base 110 can be attached to the cap 120 using adhesive, bonding, welding or similar suitable means. As will be appreciated, if the insert 150 is placed within the recess 122 in a second orientation, different from the first orientation, the alignment feature 160 will be mis-aligned with the aperture 152 and can abut against a portion of insert 150. As such the insert 150 cannot be fully received within the recess 122 and the base 110 cannot fully engage the cap 120. Advantageously, the alignment feature 160 is configured to prevent the insert 150 from being placed within the recess 122 incorrectly and can prevent the port 100 from being assembled incorrectly.

In an embodiment, the symbol 104 disposed on the lower surface of the base 110 can align with the negative symbol defined by the aperture(s) 152 of the insert 150. As such the symbol 104 can provide a visual representation of the symbol defined by the insert 150 disposed within the recess 122. In an embodiment, one or both of the base 110 and the cap 120 can be formed of an opaque material. As such, the insert 150 may not be directly observable when assembled within port 100.

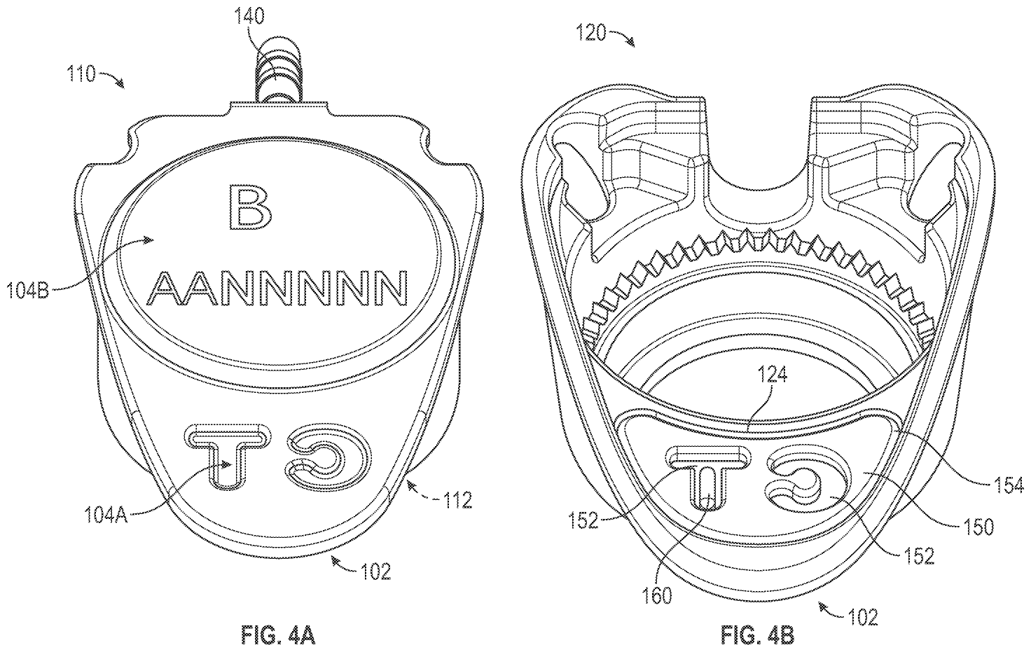

In an embodiment, as shown in FIGS. 4A-4B, the symbol 104 can include alphanumeric symbols engraved on a bottom surface of the base 110 in reverse so as to align with the symbol(s) defined by the aperture(s) 152 within the recess 122. As such, the symbol 104 can visually convey the orientation of the insert 150 within the recess 122 and a user can observe the symbol 104 to confirm the insert 150 and thereby port 100 is positioned correctly during subcutaneous placement of the port 100. In an embodiment, the base 110 can include a first symbol 104A configured to align with the insert 150 disposed within the recess 122. The base 110 can further include a second symbol 104B, as described herein, and can be configured to convey a second property of the port 100, for example a make, model, serial number, or the like, as described herein.

In an embodiment, one or both of the base 110 and the cap 120 can be formed of a transparent or translucent material to allow a user to directly observe the insert 150 disposed within the recess 122 when the port 100 is assembled. In an embodiment, one or both of the base 110 and the cap 120 can include a window formed of a transparent or translucent material and aligned with the recess 122 to allow a user to directly observe the insert 150 disposed within the recess 122 when the port 100 is assembled. Advantageously, the base 110 and the cap 120 can co-operate to provide a smooth outer profile and can contain the insert 150 therein to prevent any tissue ingrowth into the aperture 152 when the port 100 is placed subcutaneously.

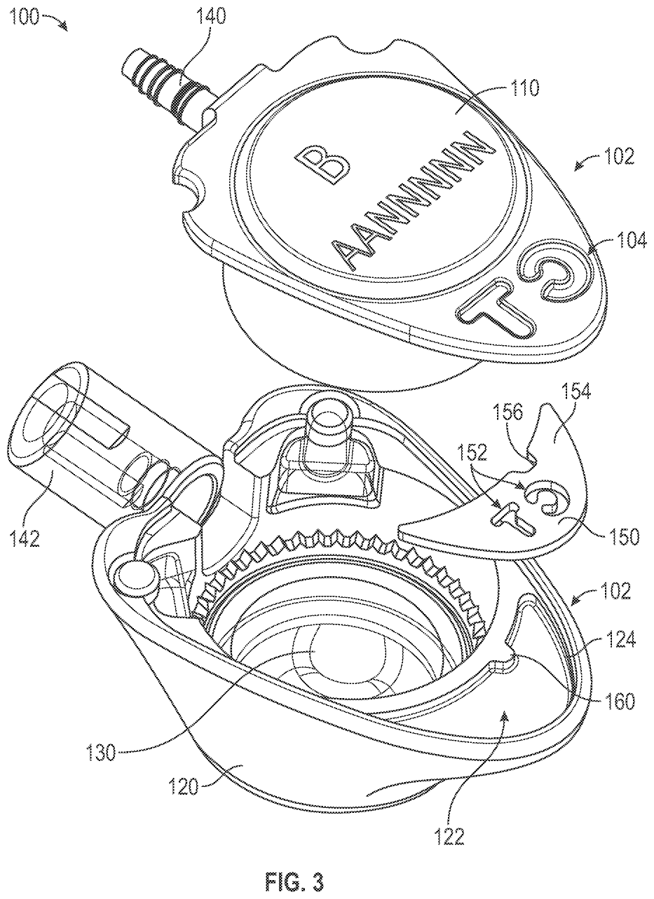

In an embodiment, as shown in FIG. 3, the alignment feature 160 can extend horizontally into the recess 122 from a perimeter 124 of the recess 122. The alignment feature 160 can be positioned along the perimeter 124 of the recess 122 to engage a notch 156 disposed in a perimeter 154 of the insert 150. As such, the alignment feature 160 can align the insert 150 with the recess 122 in a first orientation but can prevent the insert 150 from engaging the recess 122 in a second orientation, different from the first orientation, as described herein.

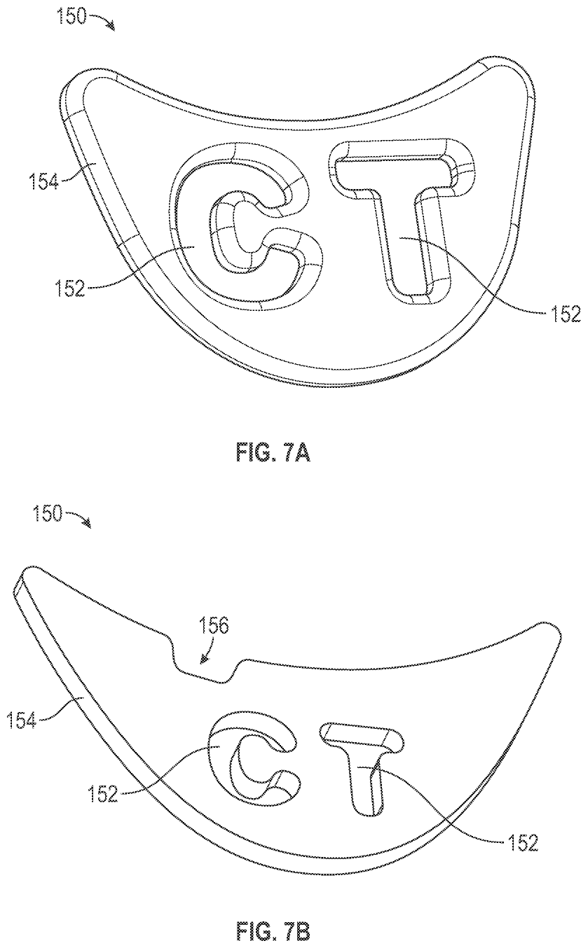

FIGS. 7A-7B show further details of exemplary inserts 150 including one or more apertures 152 and a notch 156.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous access port, comprising:
a base defining a reservoir;
a stem in fluid communication with the reservoir and extending longitudinally;
a septum positioned over the reservoir;
a cap configured to engage the base and secure the septum over the reservoir, the cap comprising a nose portion defining a recess;
an alignment feature including a protrusion extending from a surface of the recess into the recess; and
a radiopaque insert including an aperture, a perimeter of the aperture defining a symbol, the radiopaque insert configured to fit within the recess in a first orientation, the protrusion configured to maintain the radiopaque insert in the first orientation and prevent the radiopaque insert from engaging the recess in a second orientation different from the first orientation.

2. The subcutaneous access port according to claim 1, wherein the base engages the cap to secure the radiopaque insert within the recess in the first orientation.

3. The subcutaneous access port according to claim 1, wherein one of the protrusion or the base abuts against the radiopaque insert in the second orientation and prevents the cap from engaging the base.

4. The subcutaneous access port according to claim 1, wherein the protrusion is configured to engage a notch disposed in a perimeter of the radiopaque insert in the first orientation, and configured to abut against a portion of the perimeter of the radiopaque insert in the second orientation.

5. The subcutaneous access port according to claim 1, wherein the protrusion is configured to engage the aperture in the first orientation and abut against a portion of the radiopaque insert in the second orientation.

6. The subcutaneous access port according to claim 1, wherein the symbol indicates a property of the port.

7. The subcutaneous access port according to claim 1, wherein the symbol includes the letters “CT”.

8. The subcutaneous access port according to claim 1, wherein the insert is disposed on an opposite side of the reservoir from the stem, along a longitudinal axis.

9. The subcutaneous access port according to claim 1, wherein the reservoir is disposed between the stem and the insert along a longitudinal axis.

10. The subcutaneous access port according to claim 1, wherein the nose portion extends from the port along the longitudinal axis, opposite the stem.

11. A method of manufacturing a subcutaneous access port, comprising:
providing a cap including a nose portion defining a recess;
placing a radiopaque insert within the recess in a first orientation, the radiopaque insert including an aperture, a perimeter of the aperture defining a symbol;
engaging an alignment feature with the radiopaque insert, the alignment feature extending from a surface of the cap into the recess to maintain the radiopaque insert in the first orientation and prevent the radiopaque insert from engaging the recess in a second orientation different from the first orientation; and
coupling a base with the cap, a portion of the base extending across the recess to secure the radiopaque insert within the recess in the first orientation.

12. The method according to claim 11, wherein the base defines a reservoir and includes a stem extending therefrom along a longitudinal axis, the stem in fluid communication with the reservoir.

13. The method according to claim 12, further including securing a septum over the reservoir when the base is coupled with the cap.

14. The method according to claim 11, wherein one of the alignment feature or the base abuts against the radiopaque insert in the second orientation and prevents the cap from engaging the base.

15. The method according to claim 11, wherein the alignment feature engages a notch disposed in a perimeter of the radiopaque insert, the alignment feature engages the notch in the first orientation, and abuts against a portion of the perimeter of the radiopaque insert in the second orientation.

16. The method according to claim 11, wherein the alignment feature is configured to engage the aperture in the first orientation and abut against a portion of the radiopaque insert in the second orientation.

17. The method according to claim 11, wherein the symbol indicates a property of the port.

18. The method according to claim 11, wherein the symbol includes the letters “CT”.

19. The method according to claim 11, wherein the insert is disposed on an opposite side of the reservoir from the stem, along a longitudinal axis.

20. The method according to claim 11, wherein the reservoir is disposed between the stem and the insert along a longitudinal axis.

21. The method according to claim 11, wherein the nose portion extends from the port along the longitudinal axis, opposite the stem.
